(19) United States
(12) Patent Application Publication
(10) Pub. No.: US 2026/0182915 A1
(43) Pub. Date: Jul. 2, 2026

(54) COMPRESSION FOOT GARMENT WITH AN INTEGRATED BIOSENSOR SYSTEM

(71) Applicant: SickFit LLC, Arlington, TX (US)

(72) Inventor: Paden Sickles, Mt. Washington, KY (US)

(21) Appl. No.: 19/432,554

(22) Filed: Dec. 24, 2025

Related U.S. Application Data
(60) Provisional application No. 63/738,983, filed on Dec. 26, 2024.

(51) Int. Cl. A61B 5/00; A61B 5/01; A61B 5/021; A61B 5/026; A61B 5/145

(52) U.S. Cl. CPC A61B 5/6807; A61B 5/01; A61B 5/021; A61B 5/026; A61B 5/14532; A61B 2562/043

(57) ABSTRACT

A foot covering comprising an elongated fabric body. The foot covering includes a biosensor system that includes a biosensor array integrated into the elongated fabric body, and a microprocessor that is configured to removably electronically couple to the biosensor array and removably physically couple to the elongated fabric body.

### CROSS REFERENCE TO RELATED APPLICATIONS

The present application claims priority to U.S. Patent Application No. 63/738,983 that was filed on Dec. 26, 2024, entitled “Innovative Sensor Array for Proactive Prevention of Diabetic Foot Ulcers” which is hereby incorporated by reference in its entirety.

### TECHNICAL FIELD

The present disclosure generally relates to foot garments, and more particularly, foot garments that provide compression foot garments with an integrated biosensor system.

### BACKGROUND

Diabetic foot ulcers (DFUs) affect 15% of diabetic patients, contributing to 85% of diabetes-related amputations. These ulcers result in over 100,000 amputations annually in the U.S. alone and incur more than $9 billion in healthcare costs. DFUs arise due to a combination of poor circulation (peripheral artery disease), nerve damage (peripheral neuropathy), and elevated glucose levels, all of which impair the body's ability to heal wounds. Once ulcers form, they are highly prone to infection, leading to complications such as gangrene and amputation. Current treatments focus on reactive care—managing ulcers after they form through wound care, pressure off-loading, and advanced therapies such as negative pressure wound therapy (NPWT). Despite these efforts, patients remain at high risk for recurrent ulcers and subsequent amputations, highlighting the urgent need for preventive solutions.

### SUMMARY

In an embodiment, a foot covering may include an elongated fabric body. The foot covering may include a biosensor system that includes a biosensor array integrated into the elongated fabric body, and a microprocessor that is configured to removably electronically couple to the biosensor array and removably physically couple to the elongated fabric body.

One or more of the following features may be included. The biosensor array may include a plurality of biosensors distributed throughout the elongated fabric. The plurality of biosensors include one or more of: a temperature sensor; a pressure sensor; a glucose sensor; and a blood flow sensor. The microprocessor may be removably electronically coupled to the biosensor array and removably physically coupled to the elongated fabric body using a microprocessor connector assembly. The microprocessor connector assembly may include one or more magnetic couplers that removably electronically couple the microprocessor to the biosensor array and removably physically couple the microprocessor to the elongated fabric body. The microprocessor connector assembly may include a threaded portion configured to engage a corresponding threaded portion of a microprocessor housing that removably electronically couples the microprocessor to the biosensor array and removably physically couples the microprocessor to the elongated fabric body. The elongated fabric body may include a plurality of compression zones within the elongated fabric body. The plurality of compression zones include a plurality of distinct compression levels. The biosensor array may be washable.

According to another embodiment, a foot covering may include an elongated fabric body including an open end, an ankle portion, and a closed end. The foot covering may include an integrated biosensor system that includes a biosensor array integrated into the elongated fabric body, and a microprocessor that is configured to removably electronically couple to the biosensor array and removably physically couple to the elongated fabric body. The microprocessor may include a wireless communication interface for communicating wirelessly with other electronic devices.

One or more of the following features may be included. The biosensor array may include a plurality of biosensors distributed throughout the elongated fabric. The plurality of biosensors include one or more of: a temperature sensor; a pressure sensor; a glucose sensor; and a blood flow sensor. The microprocessor may be removably electronically coupled to the biosensor array and removably physically coupled to the elongated fabric body using a microprocessor connector assembly. The microprocessor connector assembly may include one or more magnetic couplers that removably electronically couple the microprocessor to the biosensor array and removably physically couple the microprocessor to the elongated fabric body. The microprocessor connector assembly may include a threaded portion configured to engage a corresponding threaded portion of a microprocessor housing that removably electronically couples the microprocessor to the biosensor array and removably physically couples the microprocessor to the elongated fabric body. The elongated fabric body may include a plurality of compression zones within the elongated fabric body. The plurality of compression zones include a plurality of distinct compression levels.

According to another embodiment, a foot covering may include an elongated fabric body including an open end, an ankle portion, and a closed end. The elongated fabric body may include a plurality of compression zones within the elongated fabric body. The foot covering may include an integrated biosensor system that includes a biosensor array integrated into the elongated fabric body, and a microprocessor that is configured to removably electronically couple to the biosensor array and removably physically couple to the elongated fabric body.

One or more of the following features may be included. The biosensor array may include a plurality of biosensors distributed throughout the elongated fabric. The plurality of biosensors include one or more of: a temperature sensor; a pressure sensor; a glucose sensor; and a blood flow sensor.

The details of one or more example implementations are set forth in the accompanying drawings and the description below. Other possible example features and/or possible example advantages will become apparent from the description, the drawings, and the claims. Some implementations may not have those possible example features and/or possible example advantages, and such possible example features and/or possible example advantages may not necessarily be required of some implementations.

### BRIEF DESCRIPTION OF THE DRAWINGS

FIG. 1 is a side perspective view of a foot garment including an integrated biosensor system, according to an example embodiment;

FIG. 2 is a side perspective view of a foot garment showing the interior of the foot garment with a biosensor array integrated into the foot garment, according to an example embodiment;

FIG. 3 is a side perspective view of a foot garment including an integrated biosensor system, according to an example embodiment;

FIG. 4 is a diagrammatic view of computer system and a biosensor management process coupled to a distributed computing network; and

FIG. 5 is a flow chart of one implementation of the biosensor management process.

Like reference symbols in the various drawings may indicate like elements.

### DETAILED DESCRIPTION OF EXAMPLE EMBODIMENTS

In general, consistent with the present disclosure, a foot garment may be a high-compression diabetic foot garment with an integrated modular biosensor system that continuously monitors multiple physiological parameters relevant to various ailments (including diabetic foot ulcers (DFUs), general circulation monitoring, sports recovery, and other neuropathy-related conditions), while simultaneously improving circulation through graduated compression. In one embodiment, the foot garment may include an elongated fabric body including an open end, an ankle portion, and a closed end; and an integrated biosensor system including: a biosensor array integrated into the elongated fabric body; and a microprocessor that is configured to removably electronically couple to the biosensor array and removably physically couple to the elongated fabric body.

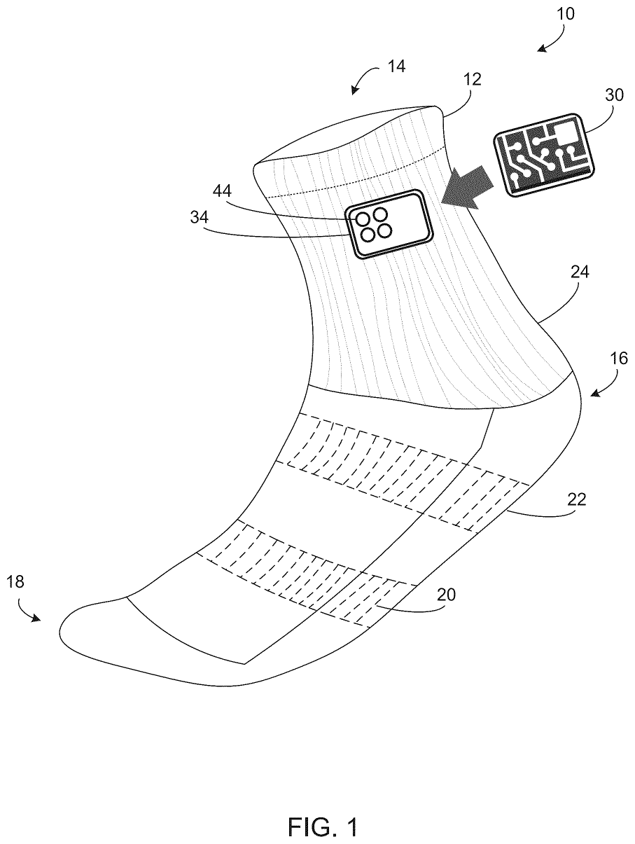

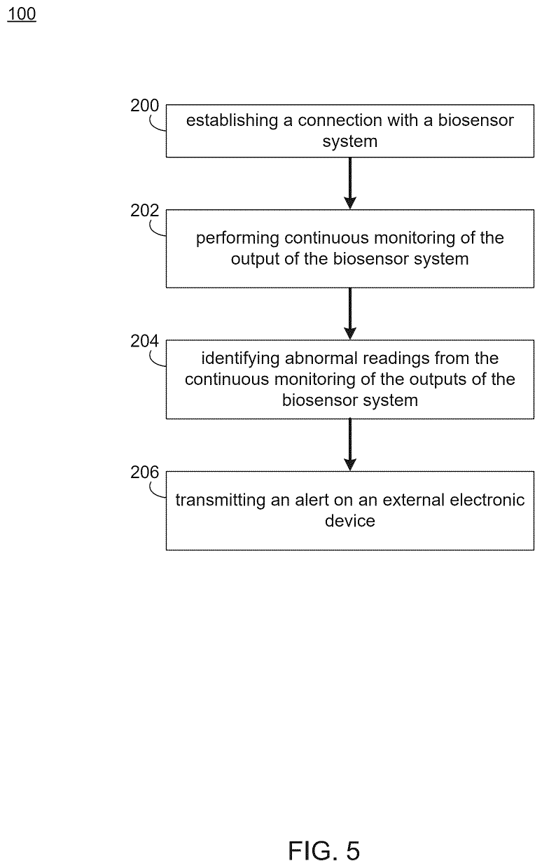

For example, and referring generally to FIGS. 1-5, a foot garment (e.g., foot garment 10) may include an elongated fabric body (e.g., elongated fabric body 12) including an open end (e.g., open end 14), an ankle portion (e.g., ankle portion 16), and a closed end (e.g., closed end 18). In some embodiments, elongated fabric body 12 may be a wrap configured to wrap around a foot using one or more fasteners to secure the elongated fabric body around a foot. Elongated fabric body 12 may be formed from a blend of natural and synthetic fibers. In some embodiments, elongated fabric body 12 is formed from materials including cotton, wool, polyester, nylon, spandex (also known as elastane or Lycra), and/or bamboo or modal. In one example, cotton is valued for its softness and breathability, while wool provides warmth and moisture-wicking properties. Synthetic fibers like polyester and nylon may add durability, elasticity, and help socks retain their shape. Spandex may be included in particular amounts (e.g., 3% to 5%, or up to 10%) to provide stretch and a snug fit. The specific blend and proportions of these materials can vary depending on the exact composition of elongated fabric body 12. In some embodiments, ankle portion 16 and/or closed end 18 may be formed from a different combination of materials than the rest of elongated fabric body 12. For example, ankle portion 16 and/or closed end 18 may include extra fabric and/or thicker fabric for greater durability from wear.

In some embodiments, the elongated fabric body includes a plurality of compression zones within the elongated fabric body. In some embodiments, the plurality of compression zones include a plurality of distinct compression levels. For example, elongated fabric body 12 may be an advanced compression knit sock with graduated compression from ankle upward to promote venous return and improve circulation in the lower extremity. In some embodiments, elongated fabric body 12 may include various compression zones (e.g., compression zones 20, 22, 24). In the example of FIG. 1, compression zone 20 is represented by a band extending around a portion of elongated fabric body 12 toward closed end 18. Compression zone 22 is represented by a band extending around a portion of elongated fabric body 12 between ankle portion 16 and closed end 18. Compression zone 24 is represented by a band extending from ankle portion 16 toward open end 14. In some embodiments, each compression zone may have distinct compression levels. As discussed above, compression zone 24 may include graduated or gradient compression that increases from ankle end 16 toward open end 12. While three examples of compression zones have been described, it will be appreciated that any number of compression zones with varying types and levels of compression may be formed within elongated fabric body 12 within the scope of the present disclosure.

In some embodiments, an integrated biosensor system (e.g., integrated biosensor system 26) includes a biosensor array (e.g., biosensor array 28) integrated into elongated fabric body 12 and a microprocessor (e.g., microprocessor 30) that is configured to removably electronically couple to biosensor array 28 and removably physically couple to elongated fabric body 12.

In some embodiments, biosensor array 28 is a distributed network of biosensors embedded into or attached onto elongated fabric body 12. Biosensor array 28 is integrated into selected portions of elongated fabric body 12. In one example, biosensor array 28 includes one or more sensor regions located at various areas associated with elevated risk of tissue breakdown in diabetic patients. In some embodiments, a heel sensor region may be disposed adjacent a heel portion of the foot, a forefoot sensor region may be disposed beneath a metatarsal head region, and a toe sensor region may be disposed beneath one or more toes.

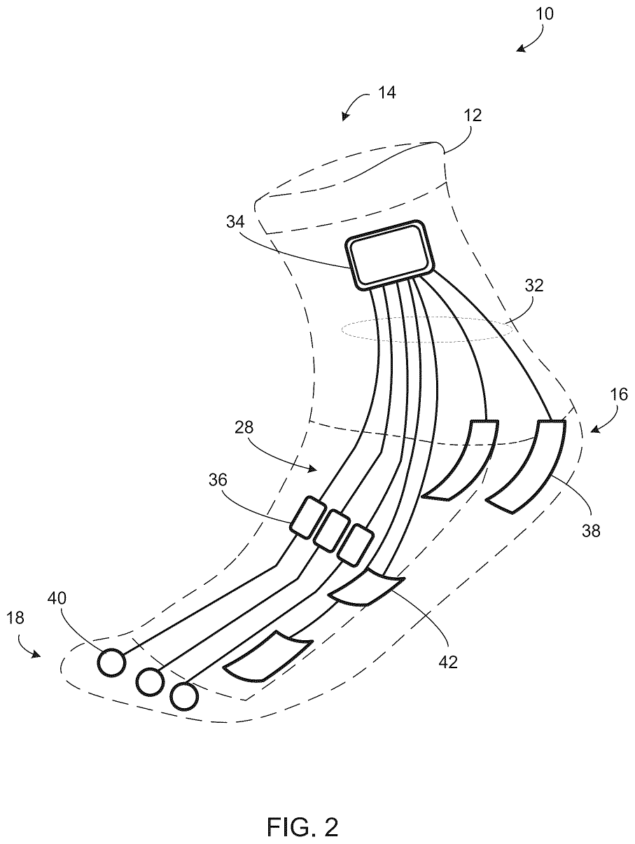

Referring also to FIG. 2, one or more conductive pathways 32 are formed in or on elongated fabric body 12. Conductive pathways 32 may include conductive yarns, printed conductive inks, flexible traces, or other textile-integrated conductors that route sensor signals from the sensor regions toward a microprocessor connector assembly (e.g., microprocessor connector assembly 34). In some embodiments, the arrangement of the sensor regions and conductive pathways 32 is configured to maintain sensor contact with the wearer's skin while preserving comfort and the functional compression characteristics of the foot garment. In some embodiments, the biosensor array is washable. For example, conductive pathways 32 and biosensors of biosensor array 28 may be sealed and waterproof such that biosensor array 28 can be washed using conventional approaches.

In some embodiments, the biosensor array includes a plurality of biosensors distributed throughout the elongated fabric. For example, biosensor array 28 may process biosensor data to provide early, real-time warning of DFU risk by tracking key indicators such as: local foot temperature; local pressure/load on high-risk areas; blood flow/perfusion proxies; and metabolic biomarkers (e.g., glucose and insulin response (via biosensors integrated into biosensor system 26)). In some embodiments, sensor “zones” are located in areas most associated with DFUs: plantar forefoot, heel, metatarsal heads, and possibly toes. The knit pattern in these zones is designed to hold sensors flush to the skin while remaining comfortable for daily wear. However, it will be appreciated that the plurality of sensors of biosensor array 28 may be positioned in various locations on or within biosensor array 28 for identifying many particular issues within the scope of the present disclosure.

In some embodiments, the plurality of biosensors include one or more of: a temperature sensor; a pressure sensor; a glucose sensor; and a blood flow sensor. For example, biosensor array 28 may include one or more temperature sensors (e.g. temperature sensors 36) which are small, flexible thermistors or similar components embedded in high-risk plantar and dorsal regions of the foot. In one example, temperature sensors 36 may be used to detect localized “hot spots” that correlate with inflammation and early ulcer formation. Biosensor array 28 may include one pressure/load sensors (e.g., pressure sensors 38) which are thin, flexible sensors integrated under the heel and forefoot. In one example, pressure sensor 38 may be used to detect prolonged high-pressure areas that may lead to tissue breakdown. Biosensor array 28 may include a glucose sensor (e.g., glucose and insulin-related sensors 40) which are chemical or electrochemical biosensors capable of detecting glucose and insulin levels in sweat, interstitial fluid, or other accessible biofluids. These may use enzyme-based sensing (e.g., glucose oxidase) or other transduction mechanisms integrated into the textile or a small interface patch. Biosensor array 28 may include one or more blood flow sensors (e.g., blood flow/perfusion sensors 42) that may infer changes in circulation indirectly (e.g., by combining temperature trends, pressure, and possibly impedance or optical signals measured at the skin). In some embodiments, each sensor is connected via conductive pathways 32 that route signals from the sensor locations to microprocessor connector assembly 34.

In some embodiments, the microprocessor is removably electronically coupled to the biosensor array and removably physically coupled to the elongated fabric body using a microprocessor connector assembly. For example, microprocessor connector assembly 34 is positioned on an exterior surface of elongated fabric body 12. In one example, microprocessor connector assembly 34 is positioned along a lateral ankle portion of elongated fabric body 12, and provides a set of electrical contacts and mechanical features for coupling to a detachable electronics module (e.g., microprocessor 30). In some embodiments, microprocessor connector assembly 34 includes a “quick-connect” design that allows microprocessor 30 to be removably coupled, reducing replacement cost (as only the elongated fabric body may need replacement), and extends the lifespan of the system.

In one example, microprocessor connector assembly 34 includes one or more magnetic couplers that removably electronically couple the microprocessor to the biosensor array and removably physically couple the microprocessor to the elongated fabric body. For example, microprocessor connector assembly may include one or more magnetic coupler portions configured to magnetically secure against corresponding metallic couple portions positioned on a housing of microprocessor 30.

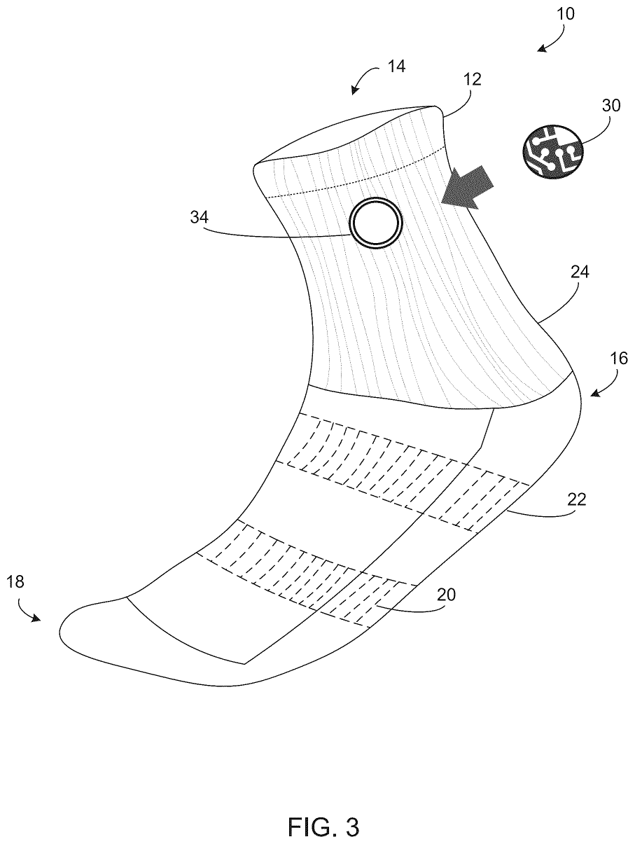

In another example, the microprocessor connector assembly includes a threaded portion configured to engage a corresponding threaded portion of a microprocessor housing that removably electronically couples the microprocessor to the biosensor array and removably physically couples the microprocessor to the elongated fabric body. Referring also to FIG. 3, microprocessor connector assembly 34 may be generally circularly shaped with a circumferentially threaded portion and microprocessor 30 may include a generally circular housing with a corresponding circumferentially threaded portion configured to engage with the threaded portion of microprocessor connector assembly 34. While two examples of particular connection mechanisms have been described, it will be appreciated that various other quick connect mechanisms (e.g., snap connectors, mechanical clips, screws, etc.) may be used within the scope of the present disclosure to secure microprocessor 30 to microprocessor connector assembly 34.

In some embodiments, microprocessor 30 may include a signal processing chip, a power source (e.g., rechargeable coin cell or flat battery); a local memory; and wireless communication interface (e.g., Bluetooth® Low Energy). For example, microprocessor 30 may read sensor data from biosensor array 28 and perform basic signal processing and aggregation. Microprocessor 30 may transmit summarized or raw data securely to an external device (e.g., a smartphone application, clinician dashboard, etc.).

In some embodiments, microprocessor 30 includes a housing that encloses one or more electronic components. For example, microprocessor 30 may be configured to receive, process, and store data from the sensors of biosensor array 28. The power source may comprise a rechargeable battery or other portable power element, and may be managed by power conditioning circuitry included in the additional circuitry. The wireless communication transceiver may be configured to transmit sensor data or derived metrics to an external device, such as a smartphone, tablet, or remote server, and may also receive configuration data or firmware updates.

The housing may include a module interface surface that carries one or more mating electrical contacts. The mating electrical contacts may be configured to electrically and mechanically couple to corresponding electrical contacts (e.g., electrical contacts 44) provided in microprocessor connector assembly 34. When microprocessor 30 is pressed into engagement with microprocessor connector assembly 34, mating electrical contacts 44 establish an electrical connection with the conductive pathways 32, thereby coupling microprocessor 30 and other electronics to biosensor array 28. In some embodiments, the housing may further include mechanical retention features, such as clips, detents, or magnetic elements, which cooperate with complementary features of microprocessor connector assembly 34 to secure microprocessor 30 in place during use while allowing removal prior to laundering the foot garment.

Although FIG. 1 illustrates a generally rectangular housing and while FIG. 3 illustrates a generally circular housing for microprocessor 30, other shapes, orientations, and component layouts may be used. For example, microprocessor 30 may be oval, or contoured to match the wearer's ankle, and may include additional components such as memory devices, charging interfaces, or indicator lights may be included within or on the housing.

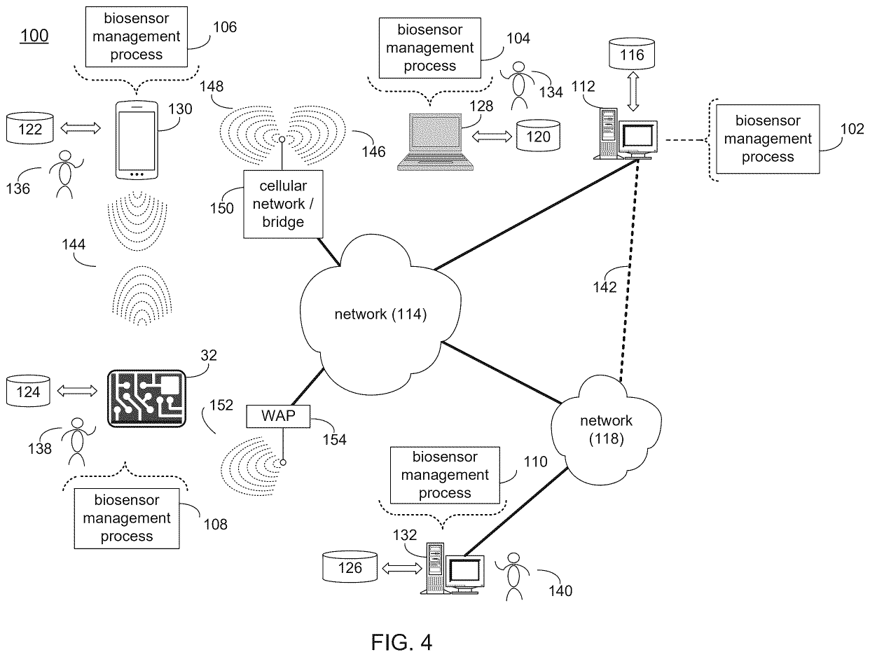

Referring to FIG. 4, there is shown biosensor management process 100. Biosensor management process 100 may be implemented as a server-side process, a client-side process, or a hybrid server-side/client-side process. For example, biosensor management process 100 may be implemented as a purely server-side process via biosensor management process 102. Alternatively, biosensor management process 100 may be implemented as a purely client-side process via one or more of biosensor management process 104, biosensor management process 106, biosensor management process 108, and biosensor management process 110. Alternatively still, biosensor management process 100 may be implemented as a hybrid server-side/client-side process via biosensor management process 102 in combination with one or more of biosensor management process 104, biosensor management process 106, biosensor management process 108, and biosensor management process 110. Accordingly, biosensor management process 100 as used in this disclosure may include any combination of biosensor management process 102, biosensor management process 104, biosensor management process 106, biosensor management process 108, and biosensor management process 110.

Biosensor management process 102 may be a server application and may reside on and may be executed by computing device 112, which may be connected to network 114 (e.g., the Internet or a local area network). Examples of computing device 112 may include, but are not limited to: a personal computer, a server computer, a series of server computers, a minicomputer, a mainframe computer, a smartphone, or a cloud-based computing platform.

The instruction sets and subroutines of biosensor management process 102, which may be stored on storage device 116 coupled to computing device 112, may be executed by one or more processors (not shown) and one or more memory architectures (not shown) included within computing device 112. Examples of storage device 116 may include but are not limited to: a hard disk drive; a RAID device; a random-access memory (RAM); a read-only memory (ROM); and all forms of flash memory storage devices.

Network 114 may be connected to one or more secondary networks (e.g., network 118), examples of which may include but are not limited to: a local area network; a wide area network; or an intranet, for example.

Examples of biosensor management processes 104, 106, 108, 110 may include but are not limited to a web browser, a game console user interface, a mobile device user interface, or a specialized application (e.g., an application running on e.g., the Android tm platform, the iOS tm platform, the Windows tm platform, the Linux tm platform or the UNIX tm platform). The instruction sets and subroutines of biosensor management processes 104, 106, 108, 110, which may be stored on storage devices 120, 122, 124, 126 (respectively) coupled to client electronic devices 10, 128, 130, 132 (respectively), may be executed by one or more processors (not shown) and one or more memory architectures (not shown) incorporated into client electronic devices 10, 128, 130, 132 (respectively). Examples of storage devices 120, 122, 124, 126 may include but are not limited to: hard disk drives; RAID devices; random access memories (RAM); read-only memories (ROM), and all forms of flash memory storage devices.

Examples of client electronic devices 10, 128, 130, 132 may include, but are not limited to, a microprocessor 30, a smartphone (not shown), a personal digital assistant (not shown), a tablet computer (not shown), laptop computer 128, smart phone 130, personal computer 132, a notebook computer (not shown), a server computer (not shown), a gaming console (not shown), and a dedicated network device (not shown). Client electronic devices 10, 128, 130, 132 may each execute an operating system, examples of which may include but are not limited to Microsoft Windows™, Android™, iOS™, Linux™, or a custom operating system.

Users 134, 136, 138, 140, may access biosensor management process 100 directly through network 114 or through secondary network 118. Further, biosensor management process 100 may be connected to network 114 through secondary network 118, as illustrated with link line 142.

The various client electronic devices (e.g., client electronic devices 10, 128, 130, 132) may be directly or indirectly coupled to network 114 (or network 118). For example, laptop computer 128 and vehicle management system 130 are shown wirelessly coupled to network 114 via wireless communication channels 146, 148 (respectively) established between laptop computer 28 and vehicle management system 130 (respectively) and cellular network/bridge 150, which is shown directly coupled to network 114. Further, microprocessor 30 is shown wirelessly coupled to network 114 via wireless communication channel 152 established between microprocessor 30 and wireless access point (i.e., WAP) 154, which is shown directly coupled to network 114. Additionally, personal computer 34 is shown directly coupled to network 118 via a hardwired network connection.

WAP 154 may be, for example, an IEEE 802.11a, 802.11b, 802.11g, 802.11n, Wi-Fi®, and/or Bluetooth® device that is capable of establishing wireless communication channel 152 between microprocessor 30 and WAP 154. As is known in the art, IEEE 802.11x specifications may use Ethernet protocol and carrier sense multiple access with collision avoidance (i.e., CSMA/CA) for path sharing. As is known in the art, Bluetooth® is a telecommunications industry specification that allows e.g., electronic devices, mobile phones, computers, and personal digital assistants to be interconnected using a short-range wireless connection.

In some embodiments, biosensor management process 10 may establish 200 a connection with a biosensor system. For example and as discussed above, biosensor management process 10 may allow an external electronic device (e.g., computing device 12) to establish 200 a connection (e.g., a wireless connection, a cellular connection, a Bluetooth® connection, etc.) with microprocessor 30. In some embodiments, biosensor management process 10 may provide an application that allows microprocessor 30 to be identified and selected for establishing a connection. In one example, biosensor management process 10 provides a login for securely accessing microprocessor 30.

In some embodiments, biosensor management process 10 may perform 202 continuous monitoring of the outputs of biosensor system 26. For example, once microprocessor 30 is attached and powered on, biosensor array 28 collects data at regular intervals (e.g., every few seconds or minutes), capturing: foot temperature at multiple zones; local pressure/time-under-pressure; trends in blood flow proxies; glucose/insulin-related data from biosensors; and/or other data from biosensors deployed on or within elongated fabric body 12.

In some embodiments, biosensor management process 10 may identify 204 abnormal readings from the continuous monitoring of the outputs of biosensor system 26. For example, biosensor management process 10 may identify 204 temperature asymmetries between feet or within regions of the same foot. In another example, biosensor management process 10 may identify 204 high-pressure durations that exceed safe thresholds. In another example, biosensor management process 10 may identify 204 trends in metabolic markers that correlate with poor glycemic control or heightened DFU risk. While examples have been provided for particular types of abnormal readings, it will be appreciated that various types of abnormal readings may be identified within the scope of the present disclosure.

In some embodiments and in response to identifying 204 abnormal readings from the continuous monitoring of the outputs of biosensor system 26, biosensor management process 10 transmits 206 an alert on an external electronic device. For example, when patterns associated with elevated DFU risk are detected (e.g., a persistent hotspot combined with sustained pressure and poor circulation indicators), biosensor management process 10 transmits 206 an alert to a user's electronic device (e.g., a smartphone) and/or transmits data to a clinician portal or electronic medical record (EMR)-connected dashboard for review. In some embodiments, biosensor management process 10 interfaces with an external electronic device's database to generate one or more remedial actions for the user based upon, at least in part, the alert. Accordingly and in some embodiments, biosensor management process 10 creates a closed data loop through early detection of abnormal readings from monitoring biosensor data, generating a user alert, recommending one or more behavioral changes (e.g., off-loading, contacting clinician), and potential reduction in ulcer formation and amputations.

Embodiments of the present disclosure provide multimodal sensing in a single foot garment. Conventional diabetic socks focus mainly on pressure redistribution and moisture wicking and do not integrate a multi-parameter sensor array. For example, other products monitor only temperature or rely on external devices rather than fully integrated textile-based sensing. Embodiments of the present disclosure combine graduated compression (to actively improve circulation) with real-time monitoring, not just passive measurement. The compression profile itself is part of the therapeutic effect, while sensors track whether the therapy is working as intended. Many wearables are either: entirely fixed and not washable, or separate devices strapped to the body. By contrast, embodiments of the present disclosure are removable and washable.

Embodiments of the present disclosure embed sensors into fabric, keeping them in high-risk zones, and using a removable microprocessor so the user can wash the foot garment as normal while preserving sensitive electronics. The foot garment in combination with biosensor management process 10 target DFU risk via combined metrics. For example, instead of just reacting to ulcers once visible, biosensor system 26 uses combined indicators: temperature trends; pressure/load; circulation proxies; and glucose/insulin information to predict DFU risk and to prompt preventive intervention. Accordingly, the foot garment is intended for day-to-day life (walking, working, standing), not only clinical visits. The sensor architecture and compression design are configured to be comfortable enough for extended use, providing continuous monitoring during the periods when risk is actually accumulating.

As will be appreciated by one skilled in the art, the present disclosure may be embodied as a method, a system, or a computer program product. Accordingly, the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present disclosure may take the form of a computer program product on a computer-usable storage medium having computer-usable program code embodied in the medium.

Any suitable computer usable or computer readable medium may be utilized. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. The computer-usable or computer-readable medium may also be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, RF, etc.

Computer program code for carrying out operations of the present disclosure may be written in an object-oriented programming language such as Java, Smalltalk, C++ or the like. However, the computer program code for carrying out operations of the present disclosure may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network/a wide area network/the Internet (e.g., network 14).

The present disclosure is described with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, may be implemented by computer program instructions. These computer program instructions may be provided to a processor of a general-purpose computer/special purpose computer/other programmable data processing apparatus, such that the instructions, which execute via the processor of the computer or other programmable data processing apparatus, create means for implementing the functions/acts specified in the flowchart and/or block diagram block or blocks.

These computer program instructions may also be stored in a computer-readable memory that may direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams in the figures may illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is

1. A foot covering comprising:
an elongated fabric body; and
a biosensor system including:
a biosensor array integrated into the elongated fabric body; and
a microprocessor that is configured to removably electronically couple to the biosensor array and removably physically couple to the elongated fabric body.

2. The foot covering of claim 1, wherein the biosensor array includes a plurality of biosensors distributed throughout the elongated fabric.

3. The foot covering of claim 2, wherein the plurality of biosensors include one or more of:
a temperature sensor;
a pressure sensor;
a glucose sensor; and
a blood flow sensor.

4. The foot covering of claim 1, wherein the microprocessor is removably electronically coupled to the biosensor array and removably physically coupled to the elongated fabric body using a microprocessor connector assembly.

5. The foot covering of claim 4, wherein the microprocessor connector assembly includes one or more magnetic couplers that removably electronically couple the microprocessor to the biosensor array and removably physically couple the microprocessor to the elongated fabric body.

6. The foot covering of claim 4, wherein the microprocessor connector assembly includes a threaded portion configured to engage a corresponding threaded portion of a microprocessor housing that removably electronically couples the microprocessor to the biosensor array and removably physically couples the microprocessor to the elongated fabric body.

7. The foot covering of claim 1, wherein the elongated fabric body includes a plurality of compression zones within the elongated fabric body.

8. The foot covering of claim 7, wherein the plurality of compression zones include a plurality of distinct compression levels.

9. The foot covering of claim 1, wherein the biosensor array is washable.

10. A foot covering comprising:
an elongated fabric body including an open end, an ankle portion, and a closed end; and
an integrated biosensor system including:
a biosensor array integrated into the elongated fabric body; and
a microprocessor that is configured to removably electronically couple to the biosensor array and removably physically couple to the elongated fabric body, wherein the microprocessor includes a wireless communication interface for communicating wirelessly with other electronic devices.

11. The foot covering of claim 10, wherein the biosensor array includes a plurality of biosensors distributed throughout the elongated fabric.

12. The foot covering of claim 11, wherein the plurality of biosensors include one or more of:
a temperature sensor;
a pressure sensor;
a glucose sensor; and
a blood flow sensor.

13. The foot covering of claim 10, wherein the microprocessor is removably electronically coupled to the biosensor array and removably physically coupled to the elongated fabric body using a microprocessor connector assembly.

14. The foot covering of claim 13, wherein the microprocessor connector assembly includes one or more magnetic couplers that removably electronically couple the microprocessor to the biosensor array and removably physically couple the microprocessor to the elongated fabric body.

15. The foot covering of claim 13, wherein the microprocessor connector assembly includes a threaded portion configured to engage a corresponding threaded portion of a microprocessor housing that removably electronically couples the microprocessor to the biosensor array and removably physically couples the microprocessor to the elongated fabric body.

16. The foot covering of claim 10, wherein the elongated fabric body includes a plurality of compression zones within the elongated fabric body.

17. The foot covering of claim 16, wherein the plurality of compression zones include a plurality of distinct compression levels.

18. A foot covering comprising:
an elongated fabric body including an open end, an ankle portion, and a closed end, wherein the elongated fabric body includes a graduated compression zone; and
an integrated biosensor system including:
a biosensor array integrated into the elongated fabric body; and
a microprocessor that is configured to removably electronically couple to the biosensor array and removably physically couple to the elongated fabric body.

19. The foot covering of claim 18, wherein the biosensor array includes a plurality of biosensors distributed throughout the elongated fabric.

20. The foot covering of claim 19, wherein the plurality of biosensors include one or more of:
a temperature sensor;
a pressure sensor;
a glucose sensor; and
a blood flow sensor.
